(19) United States
(12) Patent Application Publication
(10) Pub. No.: US 2007/0037743 A1
(43) Pub. Date: Feb. 15, 2007

(54) Novel conotoxin modulating sodium channels

(75) Inventors: Kozhalmannom Subramaniasastry Krishnan, Bangalore (IN); Padmanabhan Balaram, Bangalore (IN)

Correspondence Address: FOLEY AND LARDNER LLP;SUITE 500, 3000 K STREET NW, WASHINGTON, DC, 20007, US

(73) Assignee: NATIONAL CENTRE FOR BIOLOGICAL SCIENCES

(21) Appl. No.: 10/573,070

(22) PCT Filed: Sep. 23, 2004
(86) PCT No.: PCT/IB04/03092

§ 371 Date: Oct. 18, 2006

(30) Foreign Application Priority Data
Sep. 26, 2003 (IN) 789/CHE/2003

(51) Int. Cl. A61K 38/16; C07K 14/435

(52) U.S. Cl. 514/12; 530/324

(57) ABSTRACT

A 26 residue peptide (Am2766) with the sequence CKQAGESCDIFSQNCCVGTCAFICIE-NH₂ has been isolated and purified from the venom of the molluscivorous snail, Conus amadis, collected of the southeastern coast of India. Chemical modification and mass spectrometric studies establish that Am2766 has three disulfide bridges. Cterminal amidation has been demonstrated by mass measurements on the C-terminal fragments obtained by proteolysis. Sequence alignments establish that Am2766 belongs to the δ-conotoxin family. Am2766 inhibits the decay of the sodium current in brain rNav1.2a voltage-gated Na⁺ channel, stably expressed in Chinese hamster ovary (CHO) cells. Unlike δ-conotoxins have previously been isolated from molluscivorous snails, Am 2766 inhibits inactivation of mammalian sodium channel.

### FIELD OF INVENTION

The present invention pertains to the filed of pharmacologically useful compounds that modulate sodium channels.

### BACKGROUND

Conotoxins, a group of pharmacologically active peptides produced by diverse species of Conus snails, act with a high degree of specificity on different classes of channels and receptors in excitable cells (Myers, R. A., Cruz, L. J., Rivier, J. E. and Olivera, B. M. (1993) Chem. Rev. 93, 1923-1936; Olivera, B. M., Rivier, J., Clark, C., Ramilo, C. A., Corpuz, G. P., Abogadie, F. C., Mena, E. E., Woodward, S. R., Hillyard, D. R. and Cruz, L. J. (1990) Science 249, 257-263). The evolution of conotoxins in the venom of predator snails may be influenced by selective pressures imposed by the nature of the prey, with peptide mixtures from molluscivorous, piscivorous and vermivorous snails exhibiting differences (Olivera, B. M. (1997) Mol. Biol. Cell 8, 2101-2109). Systematic elucidation of structure-activity relationships for all components in a conotoxin mixture is impeded by the difficulties in isolating and identifying every individual peptide. Conotoxins are characterized by multiple disulfide bridges, which provide a relatively rigid peptide backbone framework, upon which amino acid side chains, important for interaction with the pharmacological receptors, are arrayed (Wakamatsu, K., Kohda, D., Hatanaka, H., Lancelin, J. M., Ishi-da, Y., Oya, M., Nakamura, H., Inagaki, F. and Sato, K. (1992) Biochemistry 31, 12577-12584). The classification of conotoxins has relied on the distribution of Cys residues in the primary sequence, the nature of the disulfide pairing topology and the functional attributes of the peptides (McIntosh, J. M., Olivera, B. M. and Cruz, L. J. (1999) Methods Enzymol. 294, 605-624; Gray, W. R. and Olivera, B. M. (1998) Annu. Rev. Biochem. 57, 665-700). As many as 14 classes of conotoxins have thus far been identified (α, αA, δ, ε, γ, κ, λ, λ/χ, μ, μO, ρ, σ, ω and ψ). The δ-conotoxins have been shown to inhibit voltage-gated Na⁺ channel inactivation. The specific role of the peptide κ PVIA in combination with a K⁺ channel antagonist κ PVIIA has been shown to be critical for prey capture in the fish-hunting snail, Conus purpurascens. Peptide combinations (cabals), which act in concert at distinct target sites, have been suggested to be important in rapid immobilization of prey (Terlau, H., Shon, K. J., Grilley, M., Stocker, M., Stuhmer, W. and Olivera, B. M. (1996) Nature 381, 148-151). The δ-conotoxins identified thus far have polypeptide chain lengths of 27-32 amino acids and have three disulfide bridges with a pattern (1-4; 2-5; 3-6), where 1 to 6 indicates the six Cys residues starting from the N-terminus. The only other class of conotoxins characterized thus far that target Na⁺ channels are the δ-conotoxins, which share a similar disulfide-bonding pattern, but have a relatively shorter polypeptide chain length of 17-22 amino acids. The isolation of δ-conotoxins from complex mixtures is rendered difficult due to their hydrophobicity.

### SUMMARY OF INVENTION

A 26 residue peptide (Am2766) with the sequence CKQAGESCDIFSQNCCVG-TCAFICIE-NH₂ has been isolated and purified from the venom of the molluscivorous snail, Conus amadis, collected of the southeastern coast of India. Chemical modification and mass spectrometric studies establish that Am2766 has three disulfide bridges. C-terminal amidation has been demonstrated by mass measurements on the C-terminal fragments obtained by proteolysis. Sequence alignments establish that Am2766 belongs to the δ-conotoxin family. Am2766 inhibits the decay of the sodium current in brain rNav1.2a voltage-gated Na⁺ channel, stably expressed in Chinese hamster ovary (CHO) cells. Unlike δ-conotoxins have previously been isolated from molluscivorous snails, Am 2766 inhibits inactivation of mammalian sodium channel.

### DETAILED DESCRIPTION OF INVENTION

The instant invention discloses a substantially pure peptide having the amino acid sequence CKQAGESCDIFSQNCCVG-TCAFICIE-NH₂ (SEQ ID NO 1).

The peptide is used a sodium channel modulator.

A process of preparing substantially pure peptide comprising of:
(i) isolation of the peptide, and
(ii) purifying the peptide by chromatographic methods.

The peptide in step (i) is isolated from venoms of Conus amadis.

The purification step (ii) is carried out by HPLC (High Performance Liquid Chromatography).

The peptide is used for treatment neurophysiological and neurological disorders.

The peptide is used for treatment neurophysiological and neurological disorders n schizophrenia, epilepsy, bipolar disorder or in syndromes that affect the nervous system.

A pharmaceutical composition comprising a peptide having the amino acid sequence CKQAGESCDIFSQNCCVG-TCAFICIE-NH₂ (SEQ ID NO 1) with or without pharmaceutically acceptable carriers.

The invention will now be discussed in the following examples, not to be considered as limiting.

### EXAMPLES

### EXAMPLE 1

Isolation of Peptide

The Conus species Conus amadis were collected from the southeastern coast of India. The glands after dissection were stored in 100% ethanol and the hydrophobic peptides extracted were subjected to high performance liquid chromatography (HPLC) purification. The alcohol extracted venom was preliminarily purified on a HP 1100 series HPLC system, using a C₁₈ reverse phase column (Zorbax, 4.6×250 mm, 5 μM particle size, 300 Å pore size). Further purification was effected on a C₁₈ reverse phase column affording higher resolution separations (Jupiter, Phenomenex, 10×250 mm, 4 μM particle size, 90 Å pore size). Water and acetonitrile containing 0.1% trifluoroacetic acid (TFA) were used as the mobile phase and a flow rate of 1.5 ml/min was maintained. Linear gradients were run from 20 to 98% acetonitrile. The absorbance was monitored at 226 nm. A large number of peaks were observed, of which Am2766 is a major peak and is quite hydrophobic as evidenced from the retention time on a C₁₈ column. Am2766 was taken up for further chemical identification.

### EXAMPLE 2

Chemical Modification

Reduction and alkylation: The purified peptide was dissolved in 30 ml, 0.1 M NH₄HCO₃ buffer, pH 8.0. For the reduction, 200 mM stock dithiothreitol (DTT) was added to a final concentration of 8 mM and incubated at 37° C. for 1.5 h. To the solution, appropriate iodoacetamide stock solution was added to get a final concentration of 40 mM and the mixture was incubated at room temperature in the dark, for 45 min. The reaction mixture was analyzed by electrospray ionization mass spectroscopy (ESIMS) through a C₁₈ column.

Acetylation: The stock acetylation reagent was prepared by mixing 20 ml acetic anhydride and 60 ml methanol. The peptide dissolved in 30 ml, 0.1 M NH₄HCO₃, pH 8.0, was mixed with 1 ml stock acetylation reagent and incubated at room temperature for 1 h. The resultant mixture was analyzed by LC-ESIMS using a C₁₈ reverse phase column.

Proteolytic digestion: The purified sample of reduced and alkylated peptide was digested with TPCK treated trypsin and TLCK treated chymotrypsin (Sigma, USA) with 10 mg of enzyme in 50 ml of NH₄HCO₃, pH 8.0 for 3 h at 37° C. The digest was directly analyzed by online LC-ESIMS.

Mass spectrometry (MS): Electrospray ionization (ESI) mass spectra were recorded using a Hewlett Packard single quadrupole mass spectrometer (HP 1100 MSD series). The samples were infused into the mass spectrometer through a reverse phase C18 column (Zorbax, 4.6×150 mm) with solvent A (0.1% acetic acid) and solvent B (acetonitrile with 0.1% acetic acid) at a flow rate of 0.25 ml/min. The data were acquired over the range m/z 50-3000 in positive ion mode and were analyzed using HP LC/MSD Chemstation software.

Matrix-assisted laser desorption and ionization time of flight (MALDI-TOF) MS analysis was carried out using a Kompact SEQ (Kratos Analytical, Manchester, UK) mass spectrometer, equipped with a nitrogen laser of wavelength 337 nm. The samples were prepared by mixing an equal amount of peptide (0.5 μl) with a matrix solution (α-cyano-4-hydroxy cinnamic acid) saturated in 0.1% TFA and acetonitrile (1:1).

The intact molecular weight of the peptide was determined using ESI and MALDI-MS. ESI-MS reveals the presence of [M+2H]²⁺ (1384 Da) and [M+3H]³⁺ (923 Da) species, which yield a molecular mass of 2766 Da. Simultaneous determination of the mass using MALDI-MS revealed a singly protonated molecule (2767 Da) along with Na⁺ and K⁺ adducts. In order to determine the number of Cys residues, the peptide was subjected to reduction with DTT and subsequently alkylated with iodoacetamide. Carboxamidomethylation yields an additional mass of 58 Da for each Cys residue. The ESIMS observed molecular mass for derivatized Am2766 was 3114 Da, showing a mass increment of 348 Da, corresponding to the presence of six Cys residues. Upon acetylation, a mass increment of 84 Da was detected, suggesting the presence of two primary amino groups, which may be tentatively assigned to a free N-terminus and a single Lys residue. The reduced and pyridylethylated peptide on conventional Edman sequencing yielded the sequence Cys-Lys-Gln-Ala-Gly-Glu-Ser-Cys-Asp-Ile-Phe-Ser-Glu-Asn-Cys-Cys-Val-Gly-Thr-Cys-Ala-Phe-Ile-Cys-Ile-Glu. The precise molecular mass detected by ESIMS was 2766 Da while the Edman sequencing results correspond to a mass of 2767 Da, assuming three disulfide bonds in the molecule. This discrepancy of 1 Da may arise due to C-terminal amidation of the peptide, a common posttranslational modification observed in many conotoxins.

Uniqueness of the sequence: CKQAGESCDIFSQNCCVG-TCAFICIE-NH₂ (SEQ ID NO 1)

Amino acid sequence: The sample was reduced with tri-n-butyl phosphine and alkylated with 4-vinyl pyridine. The pyridylethylated peptide was repurified by reverse phase HPLC and the amino acid sequence was analyzed by automated Edman degradation on a Shimadzu PPSQ-10 sequencer.

Electrophysiology: Isolated sodium currents were measured from the rat brain IIA sodium channel □-subunit (rNav1.2a), stably expressed in Chinese hamster ovary (CHO) cells (Sarkar, S. N., Adhikari, A. and Sikdar, S. K. (1995) J. Physiol. 488, 633-645). The currents were recorded using the patch clamp technique in the whole cell mode using an EPC-8 amplifier (Heka). Pipettes for patch clamp experiments were made from borosilicate glass (Clark Electromedical Instrument, UK). They were polished to give resistance of 1-3 MΩ. Solutions for patch clamp recordings were (in mM): 116 CsCl, 10 HEPES, 10 ethyleneglycol-bis-(L-aminoethylether)-N,N,NP,NP-tetraacetic acid (EGTA), 0.5 CaCl_{2;} 135 NaCl, 5 HEPES, 1 MgCl₂, and 1.5 CaCl₂, for the pipette and bath solutions, respectively, pH adjusted to 7.4 with NaOH. Data acquisition and pulse protocols were controlled with the pClamp8 software, and Digidata 1320 analog-to-digital converter (Axon Instruments Inc.). Data were low pass filtered at 3 kHz and sampled at 20 kHz. The recordings were done at 15° C. Cells were held at 380 mV. The toxin was dissolved in 50% ethanol and applied to the bath as a bolus to achieve a final concentration of 200 nM. Modification of the sodium currents was seen about 4 min after toxin application. The final alcohol concentration of 0.5% did not affect the sodium current waveform in separate experiments. Application of the Conus peptide (200 nM) resulted in marked slowing of the sodium current decay at depolarization potentials greater than +45 mV, with a slight increase in the peak sodium current.

Digestion with trypsin and chymotrypsin: In order to confirm the C-terminal amidation, the reduced and alkylated peptide was digested with the sequencing grade trypsin and chymotrypsin. The masses of the observed fragments were compared with those anticipated. It was observed that the mass of the C-terminal peptide (ICIE) was 532 Da whereas the expected value for the tetrapeptide is 533 Da, confirming C-terminal amidation.

### EXAMPLE 3

Am 2766 peptide: The sequences of ι-conotoxins, from both snail-hunting and fish-hunting snails and some selective sequences of Conus peptides exhibiting activity on Na⁺ channels are compared (Shon, K. J., Hasson, A., Spira, M. E., Cruz, L. J., Gray, W. R. and Olivera, B. M. (1994) Biochemistry 33, 11420-11425; Fainzilber, M., Lodder, J. C., Kits, K. S., Kofman, O., Vinnitsky, I., Van Rietschoten, J., Zlotkin, E. and Gordon, D. (1995) J. Biol. Chem. 270, 1123-1129; Fainzilber, M., Kofman, O., Zlotkin, E. and Gordon, D. (1994) J. Biol. Chem. 269, 2574-2580; McIntosh, J. M., Hasson, A., Spira, M. E., Gray, W. R., Li, W., Marsh, M., Hillyard, D. R. and Olivera, B. M. (1995) J. Biol. Chem. 270, 16796-16802; Fainzilber, M., Nakamura, T., Gaathon, A., Lodder, J. C., Kits, K. S., Burlingame, A. L. and Zlotkin, E. (1995) Biochemistry 34, 8649-8656; Fainzilber, M., Gordon, D., Hasson, A., Spira, M. E. and Zlot-kin, E. (1991) Eur. J. Biochem. 202, 589-595; Bulaj, G., DeLaCruz, R., Azimi-Zonooz, A., West, P., Watkins M., Yoshikami, D. and Olivera, B. M. (2001) Biochemistry 40, 13201-13208; Hill, J. M., Alewood, P. F. and Craik, D. J. (1996) Biochemistry 35, 8824-8835; Hill, J. M., Alewood, P. F. and Craik, D. J. (1996) Biochemistry 35, 8824-8835; Cruz, L. J., Gray, W. R., Olivera, B. M., Zeikus, R. D., Kerr, L., Yoshikami, D. and Moczydlowski, E. (1985) J. Biol. Chem. 260, 9280-9288). From the result, it is clear that, while the Cys framework is completely conserved across the δ-conotoxins, there is a clear grouping of the sequences, with the peptides from molluscivorous and piscivorous snails falling into distinct classes. Particularly noteworthy is the conservation of the stretch of amino acids between the second and third Cys residues in the sequences from piscivorous snails and the invariant Gly residues between the fourth and fifth Cys residues in the sequences from molluscivorous snails. It is conceivable that the nature of the target channels may influence the selection of conotoxin sequences in the predator snail. Overall differences in the distribution of both charged and hydrophobic residues are observed even within the δ-conotoxin subgroups. The δ-conotoxins isolated from Conus geographus have a much higher distribution of positive charges, shorter polypeptide chain lengths and a distinctly different pattern of distribution of Cys residues along the sequences. The μO-conotoxin Mr VIA, isolated from Conus marmoreus, has been shown to be a potent blocker of the N⁺ channel in Aplysia neurons (McIntosh, J. M., Hasson, A., Spira, M. E., Gray, W. R., Li, W., Marsh, M., Hillyard, D. R. and Olivera, B. M. (1995) J. Biol. Chem. 270, 16796-16802). Examination of the sequences shows that the Cys frameworks of the μO-conotoxin appear to resemble that of the δ-conotoxins. Further, the μO-conotoxin has a much lower net positive charge density than the δ-conotoxins, resembling the δ-conotoxins in their overall net charge. A significantly shorter conotoxin Pn IVB has been isolated from the species Conus pennaceus. Although this peptide possesses a characteristic N-terminus CC doublet, the distribution of the three C-terminal Cys residues does not appear to correspond to the pattern observed for either δ- or μ-conotoxins. This peptide has also been shown to have sodium channel blocking property (Fainzilber, M., Nakamura, T., Gaathon, A., Lodder, J. C., Kits, K. S., Burlingame, A. L. and Zlotkin, E. (1995) Biochemistry 34, 8649-8656.). The Conus peptides, which target diverse N⁺ channels, appear to vary significantly in detailed stereochemistry and surface charge distribution. This structural diversity is undoubtedly an advantage to the organism in specifically targeting various subtypes of N⁺ channels in their natural prey. Detailed structure-function studies involving specific amino acid replacements together with three-dimensional structure determination are required in order to establish a firm correlation between peptide sequence and physiological function.

### EXAMPLE 4

Process of preparing Am 2766

Purification from Conus amadis: The Conus species Conus amadis were collected from the southeastern coast of India. The glands after dissection were stored in 100% ethanol and the hydrophobic peptides extracted were subjected to high-performance liquid chromatography (HPLC) purification. The alcohol extracted venom was preliminarily purified on a HP 1100 series HPLC system, using a C₁₈ reverse phase column (Zorbax, 4.6μ250 mm, 5 WM particle size, 300 AH pore size). Further purification was effected on a C₁₈ reverse phase column affording higher resolution separations (Jupiter, Phenomenex, 10μ250 mm, 4 mM particle size, AH pore size). Water and acetonitrile containing 0.1% trifluoroacetic acid (TFA) were used as the mobile phase and a flow rate of 1.5 ml/min was maintained. Linear gradients were run from 20 to 98% acetonitrile. The absorbance was monitored at 226 nm. A large number of peaks were observed of which Am 2766 is a major peak and is quite hydrophobic as evidenced from the retention time on a C₁₈ column. This fraction is collected and purified to homogeneity by HPLC. The purified peptide is quantified by known methods, tested for electrophysiological activity and stored +4° C. for further use.

The peptide may also be obtained via the methods known to synthesize peptides. It is also possible to produce this peptide by recombinant DNA technology taking advantage of the fact that this invention describes the peptide sequence and based on which DNA sequence can be derived from the known triplet codes for each amino acid. The DNA sequence thus obtained can be synthesized/relevant gene stretch can be obtained from the snail DNA using methods such cDNA cloning, Polymerase chain reaction etc and cloned into expression vectors either in prokaryotic or eukaryotic systems. The clones thus obtained can be engineered to produce the peptide Am 2766 by known methods and purified to homogeneity by known methods.

1. A substantially pure peptide having the amino acid sequence CKQAGESCDIFSQNCCVG-TCAFICIE-NH₂ (SEQ ID NO 1).

2. A substantially pure peptide of claim 1, wherein the peptide is used a sodium channel modulator.

3. A process of preparing substantially pure peptide of claim 1, comprising of:
(i) isolation of the peptide, and
(ii) purifying the peptide by chromatographic methods.

4. A process of preparing substantially pure peptide of claim 3, wherein the peptide in step (i) is isolated from venoms of a molluscivorous snail.

5. A process of preparing substantially pure peptide of claim 4, wherein the molluscivorous snail is Conus amadis.

6. A process of preparing substantially pure peptide of claim 3, wherein the purification step (ii) is carried out by HPLC (High Performance Liquid Chromatography).

7. A substantially pure peptide of claim 1, wherein the peptide is used for treatment neurophysiological and neurological disorders.

8. A substantially pure peptide of claim 7, wherein the peptide is used for treatment neurophysiological and neurological disorders n schizophrenia, epilepsy, bipolar disorder or in syndromes that affect the nervous system.

9. A pharmaceutical composition comprising a peptide having the amino acid sequence CKQAGESCDIFSQNCCVG-TCAFICIE-NH₂ (SEQ ID NO 1) with or without pharmaceutically acceptable carriers.

10. A method of treating neurophysiological and neurological disorders including schizophrenia, epilepsy, bipolar disorder and syndromes that affect the nervous system, said method comprising step of administering pharmaceutically effective amount of peptide of SEQ ID NO: 1 or pharmaceutical composition comprising said peptide.
